(19) United States
(12) Patent Application Publication
(10) Pub. No.: US 2018/0098053 A1
(43) Pub. Date: Apr. 5, 2018

(54) IMAGING DEVICE, ENDOSCOPE APPARATUS, AND IMAGING METHOD

(71) Applicant: OLYMPUS CORPORATION, Tokyo (JP)

(72) Inventor: Shinichi IMADE, Iruma-shi (JP)

(73) Assignee: OLYMPUS CORPORATION, Tokyo (JP)

(21) Appl. No.: 15/827,598

(22) Filed: Nov. 30, 2017

Related U.S. Application Data
(63) Continuation of application No. PCT/JP2015/066073, filed on Jun. 3, 2015.

(51) Int. Cl. H04N 13/02; G02B 23/26; G02B 23/24; A61B 1/00; A61B 1/04; G01C 3/14

(52) U.S. Cl. CPC H04N 13/0289; H04N 13/0257; G02B 23/26; G02B 23/2484; G01C 3/14; A61B 1/04; A61B 1/00193; A61B 1/00186; A61B 1/00006

(57) ABSTRACT

An imaging device includes: an image sensor; an imaging optical system; a movable mask; and a second filter. The imaging optical system forms an image of a subject on the image sensor, by using a first optical path and a second optical path involving parallax relative to the first optical path. The movable mask includes a first filter transmitting light in a first wavelength band and a light shielding section, and is movable relative to the imaging optical device. The second filter transmitting light in a second wavelength band different from the first wavelength band is provided in the second optical path.

### CROSS REFERENCE TO RELATED APPLICATION

This application is a continuation of International Patent Application No. PCT/JP2015/066073, having an international filing date of Jun. 3, 2015, which designated the United States, the entirety of which is incorporated herein by reference.

### BACKGROUND

Techniques for optically measuring a three-dimensional shape have conventionally been known, with various methods for the measuring proposed. The proposed methods include: stereoscopic imaging based on a stereoscopic view with both left and right eyes; phase shift by patterned illumination using a sinusoidal pattern and the like; and Time of Flight (TOF) based on time measurement for reflected light.

The stereoscopic imaging can be achieved with a simple mechanism with a stereoscopic optical system used for an imaging system, and thus requires no special illumination mechanisms or illumination control, and also requires no advanced signal processing. Thus, this technique can be suitably implemented in a small space and thus is advantageous in an imaging system that has been progressively downsized recently. For example, the technique can be applied to an end of an endoscope apparatus, to a visual sensor in a small robot, and for various other needs. Such an application is likely to require not only a highly accurate measurement function but also a normal observation function with high image quality. Thus, to ensure a sufficient resolution, it is a common practice to form parallax images on a common image sensor instead of using separate image sensors. The basic idea of the stereoscopic imaging is to obtain a distance to a subject based on an amount of parallax between left and right images. If the left and right images fail to be separately formed on the common image sensor, the amount of parallax cannot be detected, and thus the distance information cannot be obtained.

JP-A-2010-128354 discloses an example of a method of separately forming left and right images. Specifically, switching between left and right imaging optical paths is performed along time with a mechanical shutter, so that the left and the right images are obtained in a time-division manner JP-A-2013-3159 discloses another method in this context. Specifically, an RG filter is inserted in the left half of a single imaging optical path, and a GB filter is inserted in the right half of the path, so that left and right images are separately obtained based on an R image and a B image in a captured image. In JP-A-2013-3159, an observation image is acquired in a normal observation mode, with the RG filter and the GB filter retracted from the imaging optical paths.

### SUMMARY

According to one aspect of the invention, there is provided an imaging device comprising: an image sensor;

an imaging optical device forming an image of a subject on the image sensor by using a first optical path and a second optical path involving parallax with respect to the first optical path;

a movable mask including a first filter transmitting light in a first wavelength band and a light shielding section, the movable mask being movable relative to the imaging optical device; and

a second filter transmitting light in a second wavelength band different from the first wavelength band, the second filter being provided to the second optical path

According to another aspect of the invention, there is provided an imaging device comprising: an image sensor;

an imaging optical device forming an image of a subject on the image sensor by using a first optical path and a second optical path involving parallax with respect to the first optical path;

a movable mask including a first filter transmitting light in a first wavelength band, a second filter transmitting light in a second wavelength band, and a light shielding section, the movable mask being movable relative to the imaging optical device

According to another aspect of the invention, there is provided an endoscope apparatus comprising the imaging device as defined in any of the above

According to another aspect of the invention, there is provided an imaging method comprising: setting, in a non-stereoscopic mode, a movable mask including a first filter transmitting light in a first wavelength band and a light shielding section in a first state, in such a manner that the first filter is not inserted in a first optical path of an imaging optical device and the light shielding section is inserted in a second optical path of the imaging optical device, the second optical path involving parallax with respect to the first optical path;

and setting, in a stereoscopic mode, the movable mask to be in a second state in such a manner that the first filter is inserted in the first optical path and the light shielding section is not inserted in the second optical path.

### BRIEF DESCRIPTION OF THE DRAWINGS

FIG. 1 illustrates an example of a basic configuration according to one embodiment.

FIG. 2 further illustrates the example of the basic configuration according to the embodiment. FIG. 3 illustrates an example of a detailed configuration of a fixed mask and a movable mask.

FIG. 4 further illustrates the example of the detailed configuration of the fixed mask and the movable mask.

FIG. 5 illustrates spectral characteristics of a left-eye optical path of the fixed mask, spectral characteristics of a right-eye optical path of the fixed mask, and spectral characteristics of a left-eye optical path of the movable mask.

FIG. 6 illustrates spectral characteristics of a captured image obtained in an observation mode.

FIG. 7 illustrates spectral characteristics of a captured image obtained in a stereoscopic measurement mode.

FIG. 8 illustrates a modification of the fixed mask and the movable mask.

FIG. 9 illustrates a modification of the fixed mask and the movable mask.

FIG. 10 illustrates a modification of an imaging optical system.

FIG. 11 illustrates a modification of the imaging optical system.

FIG. 12 illustrates the principle of stereoscopic measurement.

FIG. 13 illustrates a configuration example of an endoscope apparatus according to the embodiment.

FIG. 14 illustrates a sequence of switching between the observation mode and the stereoscopic measurement mode.

FIG. 15 illustrates countermeasure against movement of a subject or an imaging system.

FIG. 16 illustrates an example of a second configuration of the endoscope apparatus according to the embodiment.

### DESCRIPTION OF EXEMPLARY EMBODIMENTS

Stereoscopic imaging is negatively affected by a movement of an imaging system or a subject. For example, in JP-A-2010-128354, right and left images are captured in a time-division manner Thus, the movement of the imaging system or the subject results in detection of a phase difference including a shifted amount due to the movement. This results in a measurement error because the phase difference is difficult to be separated into the shifted amount and the actual phase difference. In JP-A-2013-3159, image capturing is switched between that for an observation image and that for a parallax image. However, this technique is designed for autofocusing, and is not designed for high speed switching required for an observation image and a parallax image to match in the three-dimensional shape measurement. The configuration in JP-A-2013-3159 includes two movable sections, and thus requires a large driving mechanism and involves a higher risk of failure or other like disadvantages.

For example, in an application, such as an endoscope apparatus, where a camera is not fixed relative to a subject, the relative movement between the imaging system and the subject is likely to have a negative impact. In other words, if the movement can occur without imposing any negative impact, shape measurement with a moving camera or other like measurement, which has been difficult in the conventional techniques, might be achievable.

Some aspects of the present embodiment can provide an imaging device, an endoscope apparatus, an imaging method, and the like with which stereoscopic measurement can be performed and an observation image can be captured while being less affected by the movement of the imaging system or a subject.

According to one embodiment of the invention, there is provided an imaging device comprising: an image sensor;

an imaging optical device forming an image of a subject on the image sensor by using a first optical path and a second optical path involving parallax with respect to the first optical path;

a movable mask including a first filter transmitting light in a first wavelength band and a light shielding section, the movable mask being movable relative to the imaging optical device; and

a second filter transmitting light in a second wavelength band different from the first wavelength band, the second filter being provided to the second optical path

According to one aspect of the present embodiment, the movable mask is movable relative to the imaging optical device. The stereoscopic measurement can be performed and an observation image can be captured with the position of the movable mask switched. For example, two optical paths are provided so that stereoscopic imagine can be performed in a non-time-division manner, and a single movable mask is used as the movable section. Thus, the negative impact due to the movement of the imaging system or the subject can be reduced.

The present embodiment will be described below. The present embodiment described below does not unduly limit the scope of the present invention described in the appended claims. Not all the components described in the present embodiment are required to embody the present invention.

In the description below, an example where the present invention is applied to an industrial endoscope apparatus is described. However, the application of the present invention is not limited to industrial endoscope apparatuses. The present invention may be applied to any three-dimensional measurement device that measures a three-dimensional shape through stereoscopic imaging (a method of acquiring distance information on a subject by detecting a phase difference between two images obtained with an imaging system involving parallax), and to any imaging device having a three-dimensional measurement function (such as a medical endoscope apparatus, a microscope, an industrial camera, and a visual function of a robot, for example).

### 1. Basic Configuration

First of all, an overview of the present embodiment is described, and then a basic configuration (principle configuration) according to one embodiment is described.

For example, an examination using an endoscope apparatus is performed as follows. A scope is inserted into an examination target to check whether there is an abnormality while capturing normal images. When a portion, such as a scar, to be observed in detail is found, the three-dimensional shape of the portion is measured to determine whether a further examination is required. Thus, the normal observation image is captured with white light. For example, stereoscopic imaging may be performed with white light so that stereoscopic measurement and the image capturing with white light can both be achieved. The stereoscopic imaging using white light requires an image sensor to be divided into left and right regions, and a left image and a right image to be respectively formed on the left and the right regions. Thus, only an image with a low resolution can be obtained. A color phase difference method may be employed to form the left and the right images on a single region of the image sensor. Unfortunately, this method results in a captured image with color misregistration that is unacceptable as the observation image.

In view of the above, time-division switching (for example, JP-A-2010-128354) is required for forming the left and the right images on the single region of the image sensor with white light. However, relative movement between an imaging system and a subject leads to shifting due to the movement between the left and the right images, resulting in inaccurate triangulation. Devices such as endoscope cannot have a camera fixed relative to the subject and thus are highly likely to involve this shifting due to movement.

In the present embodiment, an observation image with high resolution can be captured with white light, and the stereoscopic measurement in a non-time-division manner can be performed based on the color phase difference method.

JP-A-2013-3159 described above discloses an example of performing the stereoscopic measurement in a non-time-division manner based on the color phase difference method. However, the configuration in JP-A-2013-3159 employs the stereoscopic measurement for autofocusing, and thus it is reasonable to believe that the configuration is not designed for high speed switching between the mode for observation image and the mode for stereoscopic measurement. Furthermore, the configuration includes two filters as movable sections, and thus is unsuitable for the high speed switching in the first place.

Furthermore, in the configuration in JP-A-2013-3159, a single optical path is simply divided into left and right sides at the center, and thus is difficult to ensure a sufficient distance between pupils. Thus, accuracy of the distance measurement is difficult to improve. The endoscope apparatus needs to perform panning and focusing, and thus has a small aperture stop (large F value). Logically, dividing a small diameter of such an aperture stop into left and right sides is likely to result in a short distance between the pupils.

The time-division switching, including time-division switching between left and right for stereoscopic imaging, requires mechanical (switching) motion of a shutter and a spectral filter. The mechanical motion inherently involves a risk of error and failure, and the switched states (positions) of the shutter and the spectral filter need to be detected, and correction is required when an error is found. When such a detection function is implemented, the detection and correction are easier with a smaller variety of errors involved. In this context, the configuration in JP-A-2013-3159 is difficult to guarantee the detection and the correction because the configuration involves a risk of various types of errors and example of which includes one or both of the two spectral filters failing to be inserted in the pupil.

The present embodiment can overcome these problems described above with the following configuration. More specifically, a feature of the configuration is that subject images, formed with left-eye and right-eye optical systems, are formed on a common region of a signal image sensor. The configuration includes a switching mechanism so that high speed alternate switching between a left-eye optical path (first optical path) and a right-eye optical path (second optical path) can be achieved, and performs time-division switching between an observation mode, in which a first image (observation image) is acquired, and a measurement mode, in which a second image (parallax image, stereoscopic image, left and right images, or measurement image) is acquired.

The switching mechanism is set in such a manner that the first image, used for a normal observation, is obtained only with the left-eye optical path, and that the second image, used for measurement, is obtained with images formed with the left-eye optical path and the right-eye optical path overlapped with each other. Spectral filters are provided in the optical paths so that the left-eye image and the right-eye image correspond to separate wavelength bands.

Generally, the observation image is a normal color image involving no parallax, whereas the measurement image includes left and right separate parallax images. Three-dimensional information is acquired by obtaining an amount of parallax by using separate images, and then calculating distance information, indicating a distance to the subject, based on a principle of the stereoscopic measurement. In the measurement mode, the parallax images can be simultaneously obtained, and thus the system is free of a measurement error factor due to movement of the subject or the imaging system. As described later, the imaging system with the left-eye optical path and the right-eye optical path as separate paths can be implemented with a single movable section, and thus can achieve high speed switching, smaller size, error detection, and the like. The imaging system with the left-eye optical path and the right-eye optical path as separate paths can be downsized while ensuring the parallax, and can achieve higher measurement accuracy. As described later, the observation image may include a near infrared color region, so that only a near infrared image can be extracted through calculation with the observation image and the measurement image.

An application of the present invention includes a device having an imaging system that is not stably positioned (fixed) and having an imaging mechanism too small to use a large image sensor for ensuring a sufficient resolution. A typical example of such a device includes an industrial endoscope. Still, the application of the present invention is not limited to such a device, and the present invention can be widely applied to a three-dimensional measurement device directed to high-resolution monitoring and highly accurate measurement.

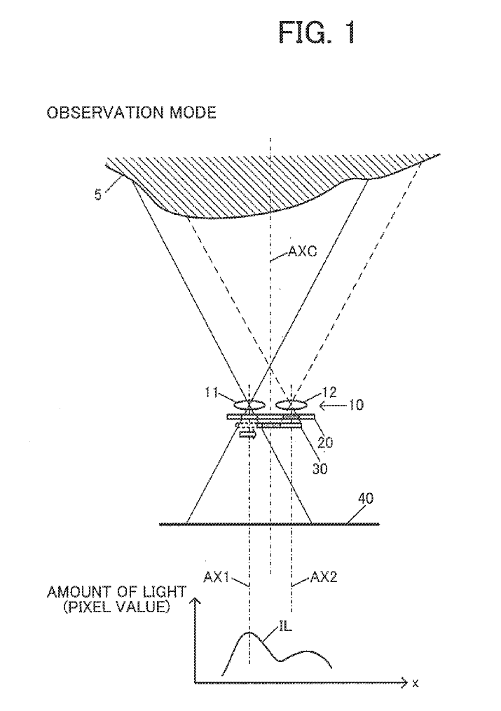

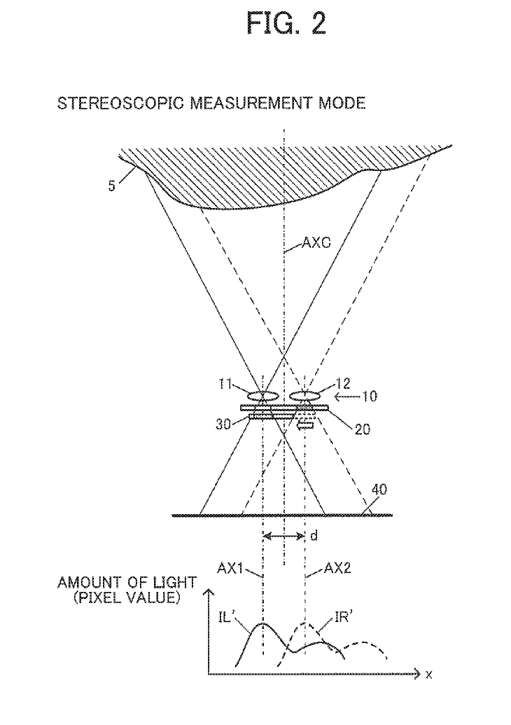

Now, the basic configuration according to the present embodiment is described with reference to FIG. 1 and FIG. 2. FIG. 1 and FIG. 2 each include a cross-sectional view of an imaging section as viewed in a lateral direction (on a plane including an optical axis) and a graph illustrating a relationship between an amount of light focused on the image sensor (or a pixel value of an image formed on the image sensor) and a position x. The position x is a position (coordinate) in a direction orthogonal to the optical axis of the imaging optical system, and is a pixel position of the image sensor for example. Although the position is actually defined in a two-dimensional coordinate system, the position is described based on a one-dimensional coordinate system corresponding to a parallax direction in the two dimensional coordinate system.

The endoscope apparatus according to the present embodiment includes: an imaging optical system 10; a movable mask 30 (first mask); a fixed mask 20 (second mask); and an image sensor 40. The imaging optical system 10 includes a left-eye imaging system 11 (first imaging optical system) and a right-eye imaging system 12 (second imaging optical system). In the example described herein, the image sensor 40 includes a color filter with RGB Bayer arrangement. However, this should not be construed in a limiting sense. For example, a complementary color filter or the like may be provided.

As illustrated in FIG. 1 and FIG. 2, reflected light from a subject 5 passes through the imaging optical system 10 including two systems (the left-eye imaging system 11 and the right-eye imaging system 12) so that an image is formed on a single image sensor 40 based on the light. A light emitting mechanism that emits light onto the subject 5 is omitted in the figure. In the figure, d represents a distance between an optical axis AX1 of the left-eye imaging system 11 and an optical axis AX2 of the right-eye imaging system 12, serving as a baseline length in the stereoscopic measurement. A straight line AXC is in parallel with the optical axes AX1 and AX2 in a plane including the optical axes AX1 and AX2, and serves as the parallax center in the stereoscopic measurement.

The left-eye imaging system 11 and the right-eye imaging system 12 each include an imaging lens. For example, the fixed mask 20 and the movable mask 30 are disposed at a pupil position of the imaging system, and may be disposed more on the image side than the imaging system. The fixed mask 20 is fixed with respect to the imaging system, whereas the movable mask 30 can have the position switched on a plane orthogonal to the optical axes AX1 and AX2. Thus, the movable mask 30 can be in a first state illustrated in FIG. 1, corresponding to the observation mode (first mode, non-stereoscopic mode, a single optical system mode), and in a second state, corresponding to a stereoscopic measurement mode (second mode, stereoscopic mode) illustrated in FIG. 2. These two modes can be switched from one to another at high speed.

The fixed mask 20 includes: a plate-shaped light shielding section (light shielding member) provided with two stop holes (a left-eye stop hole and a right-eye stop hole); and a spectral filter provided to the right-eye stop hole. The optical axis AX1 passes through the left-eye stop hole (through the center of the circle of the hole for example), and the optical axis AX2 passes through the right-eye stop hole (through the center of the circle of the hole for example). A portion other than the stop hole is covered with the light shielding section, and thus light cannot pass through this portion. For example, the left-eye stop hole may be a through hole, or may be provided with a spectral filter of some sort (for example, a broadband spectral filter at least transmitting white light).

The movable mask 30 includes: a light shielding section (light shielding member), having a plate shape, provided with a single stop hole (left-eye stop hole); and a spectral filter provided to the left-eye stop hole. The size of the movable mask 30 is set in such a manner that one of the two stop holes of the fixed mask 20 can be open (does not overlap with the movable mask 30) in each of the observation mode (normal observation mode) and the stereoscopic measurement mode. FIG. 1 and FIG. 2 each illustrate a configuration where the movable mask 30 is disposed more on the image side than the fixed mask 20. Alternatively, the movable mask 30 may be disposed more on the object side than the fixed mask 20.

The spectral characteristics of the left-eye stop hole of the fixed mask 20 are hereinafter denoted with FL, the spectral characteristics of the right-eye stop hole are hereinafter denoted with FR, and the spectral characteristics of the left-eye stop hole of a movable spectral mask are hereinafter denoted with SL For the sake of understanding, the spectral filters provided to the stop holes are also denoted with the signs FL, FR, and SL.

FIG. 1 illustrates the state corresponding to the observation mode. In this state, the left-eye side optical path is open through the left-eye stop hole of the fixed mask 20, and the right-eye side optical path is closed (shielded) by the movable mask 30. Thus, an image IL formed on the image sensor 40 is obtained with the left-eye imaging system 11 only, whereby a normal captured image (obtained with a single optical system and white light) is obtained.

FIG. 2 illustrates the state corresponding to the stereoscopic measurement mode. In this state, the left-eye stop hole of the fixed mask 20 and the left-eye stop hole of the movable mask 30 overlap with each other along the optical axis AX1. Thus, in the left-eye side optical path, light that has passed through the right-eye stop hole of the fixed mask 20 is filtered by the short-wavelength (blue) spectral filter SL (first filter) of the movable mask 30. Thus, an image IL′ including short wavelength components is formed on the image sensor 40. In the right-eye side optical path that is not shield by the movable mask 30, light for image forming is filtered by the long-wavelength (red) spectral filter FR (second filter) of the fixed mask 20. Thus, an image IR′ including long-wavelength components is formed on the same image sensor 40.

In this manner, in the stereoscopic measurement mode, the image which is the short-wavelength image obtained with blue pixels in the image sensor 40, and the image IR′, which is the long-wavelength image obtained with red pixels in the image sensor 40, can be separately acquired through the two optical paths. Thus, in the stereoscopic measurement mode, the left-eye image IL′ and the right-eye image IR′, with a phase difference, can be simultaneously and separately obtained, whereby the stereoscopic measurement can be performed with the phase difference images.

### 2. Fixed Mask and Movable Mask

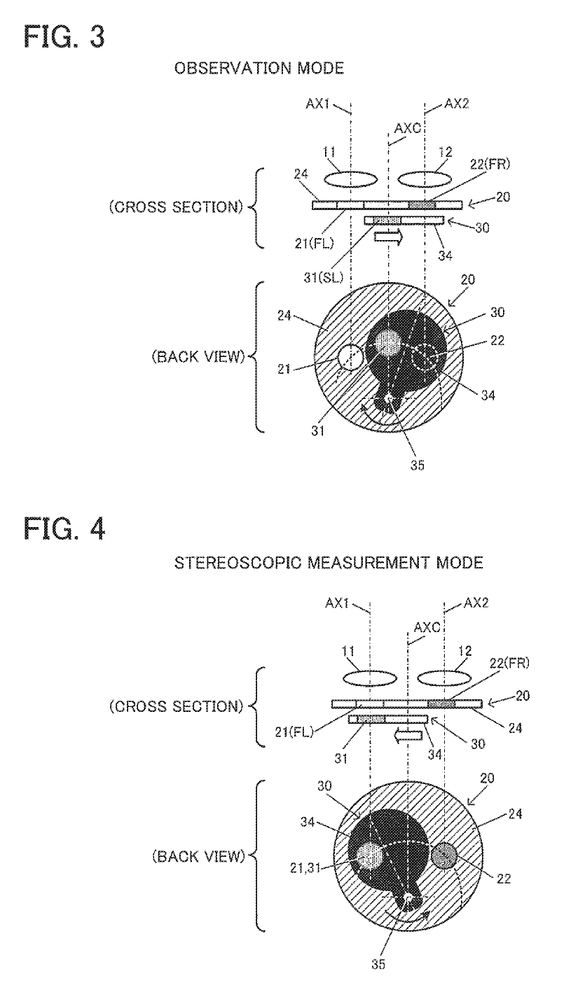

FIG. 3 and FIG. 4 illustrate detail configuration examples of the fixed mask 20 and the movable mask 30. FIG. 3 and FIG. 4 each include a cross-sectional view of the imaging optical system 10, the fixed mask 20, and the movable mask 30, and a diagram illustrating the fixed mask 20 and the movable mask 30 as viewed in the optical axis direction (a back view as viewed from the image side).

The left-eye optical path of the fixed mask 20 has a stop hole 21 (through hole) in an open state, and the right-eye optical path has a stop hole 22 including the long-wavelength spectral filter FR. The stop holes 21 and 22 are holes with sizes corresponding to the depth of field required for the imaging system for example (for example, circular holes with a size defined with a diameter), and are formed in a light shielding section 24 (light shielding member). The stop holes 21 and 22 have the centers (the center of a circle for example) respectively matching (or substantially matching) the optical axes AX1 and AX2. The light shielding section 24 is a plate-shaped member provided to be orthogonal with respect to the optical axes AX1 and AX2 for example, to shield a casing, including the optical systems 11 and 12, in front view (or back view) of the casing.

The movable mask 30 includes: a stop hole 31 provided with the short-wavelength spectral filter SL; and a light shielding section 34 (light shielding member) provided with the stop hole 31. For example, the stop hole 31 is slightly larger than the stop hole 21 of the fixed mask 20, or may be a hole with a size corresponding to the depth of field required for the imaging system (for example, a circular hole with a size defined by a diameter). In the stereoscopic observation mode, the stop hole 31 has the center (for example, the center of the circle) matching (or substantially matching) the optical axis AX1. The light shielding section 34 is connected to a rotational shaft 35 orthogonal to the optical axes AX1 and AX2, and is a plate-shaped member provided to be orthogonal to the optical axes AX1 and AX2 for example. The light shielding section 34 has a form obtained with a plate for connection to the rotational shaft 35 extending from a circular plate, for example. However, this should not be construed in a limiting sense, and any shape may be employed as long as the states illustrated in FIG. 3 and FIG. 4 can be established.

The movable mask 30 rotates about the rotational shaft 35 by a predetermined angle in the direction orthogonal to the optical axes AX1 and AX2. For example, this rotational motion can be implemented with a piezoelectric element, a motor, or the like. In the observation mode illustrated in FIG. 3, the left-eye optical path (stop hole 21) of the fixed mask 20 is in the open state and the right-eye optical path (stop hole 22) is in a shielded state, as a result of the rotation and inclination of the movable mask 30 toward the right-eye side by the predetermined angle. In the stereoscopic measurement mode illustrated in FIG. 4 as a result of the rotation and inclination of the movable mask 30 toward the left-eye side by the predetermined angle, the spectral filter (stop hole 31) of the movable mask 30 overlaps with the left-eye optical path (stop hole 21) of the fixed mask 20 so that only the short-wavelength components can pass through, and also the shielding of the right-eye optical path (stop hole 22) is released. The stop hole 22 including the long-wavelength spectral filter FR of the fixed mask 20 is exposed so that only the long-wavelength components can pass through.

In the description above, the two states are established with the movable mask 30 rotated by the predetermined angle about the shaft. However, this should not be construed in a limiting sense. For example, the two states may be established with a sliding motion of the movable mask 30. For example, the rotational motion or the sliding motion can be implemented with a magnet mechanism, a piezoelectric mechanism, or the like that may be appropriately selected to achieve a high speed motion and high durability.

### 3. Spectral Filter Characteristics of Left-Eye Optical Path and Right-Eye Optical Path

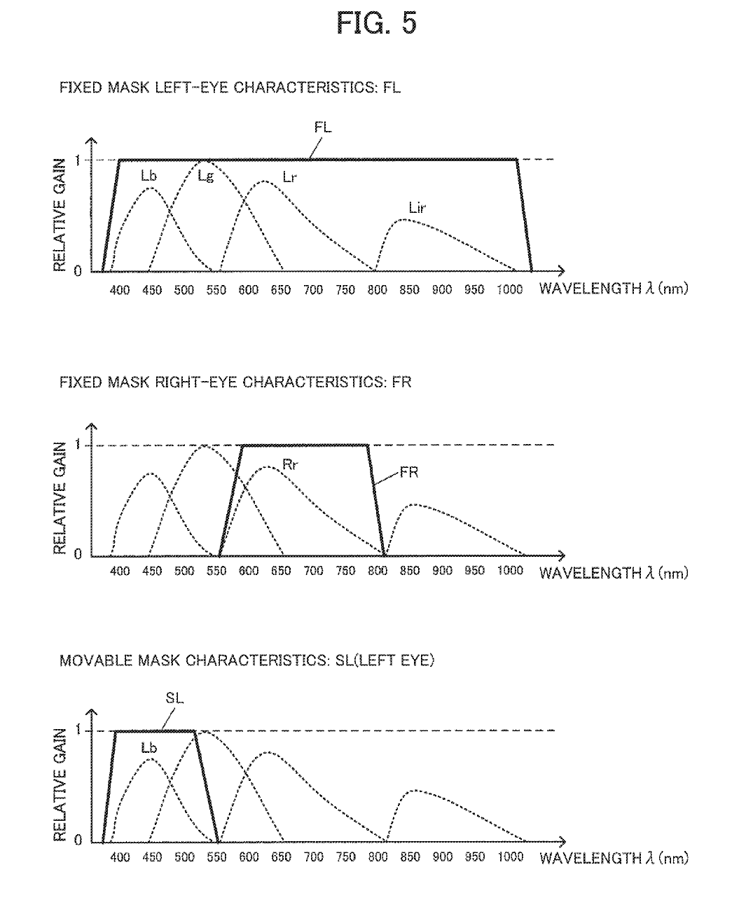

FIG. 5 illustrates the spectral characteristics FL of the left-eye optical path of the fixed mask 20, the spectral characteristics FR of the right-eye optical path of the fixed mask 20, and the spectral characteristics SL of the left-eye optical path of the movable mask 30. In FIG. 5, a relative gain represents relationship between a transmittable wavelength and a transmittance of the spectral filter (or the through hole). A dotted line represents the spectral characteristics (spectral sensitivity characteristics) of color pixels in the image sensor 40, as reference characteristics. Signs “L” and “R” respectively represent the left-eye optical path and the right-eye optical path. Signs “r”, “g”, “b”, and “ir” respectively represent a red color, a green color, a blue color, and near infrared. For example, “Lb” represents the spectral characteristics of light that passes through the left-eye optical path to be detected by blue pixels of the image sensor 40. For the sake of understanding, each of images obtained with these spectral characteristics is also denoted with the corresponding sign (Lb or the like).

As illustrated in FIG. 5, the spectral characteristics FL of the left-eye optical path of the fixed mask 20 includes all the spectral characteristics Lb, Lg, Lr, and Lir of the color pixels of the image sensor 40. These spectral characteristics may be set for the light emitted onto the subject 5 in a simple open state (through hole). Alternatively, the spectral filter having the spectral characteristics FL illustrated in FIG. 5 may be provided to the left-eye stop hole 21.

The spectral characteristics FR of the right-eye optical path of the fixed mask include the spectral characteristics Rr of the red color r but do not include the spectral characteristics of the blue color b. Note that the spectral characteristics FR need not to be completely different from the spectral characteristics of the blue color b or may not include the spectral characteristics Rr of red color r entirely, as long as the separation between the left and the right images (the red image and the blue image) can be sufficiently ensured.

The spectral characteristics SL of the left-eye optical path of the movable mask 30 in the stereoscopic measurement mode include the spectral characteristics Lb of the blue color b but do not include the spectral characteristics of the red color r. Note that the spectral characteristics SL need not to be completely different from the spectral characteristics of the red color r or may not include the spectral characteristics Lb of blue color b entirely, as long as the separation between the left and the right images (the red image and the blue image) can be sufficiently ensured. The right-eye optical path of the movable mask 30 in the stereoscopic measurement mode is achieved by simply achieving the open state of the right-eye optical path of the fixed mask 20, and thus is not limited to particular spectral characteristics.

### 4. Captured Image

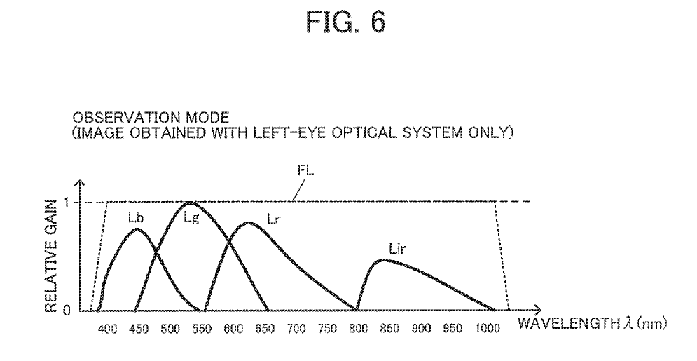

FIG. 6 illustrates spectral characteristics of a captured image, obtained with the image sensor 40 through the left-eye optical path during the observation mode. In FIG. 6, the solid line represents the spectral characteristics of the captured image, and the dotted line represents the spectral characteristics FL of the left-eye optical path. In the observation mode, the captured image is acquired only through the left-eye optical path, and thus includes the components of the red color r, the green color g, the blue color b, and near infrared ir. Thus, an image captured with a single optical system, with no parallax image superimposed thereon, can be simply obtained.

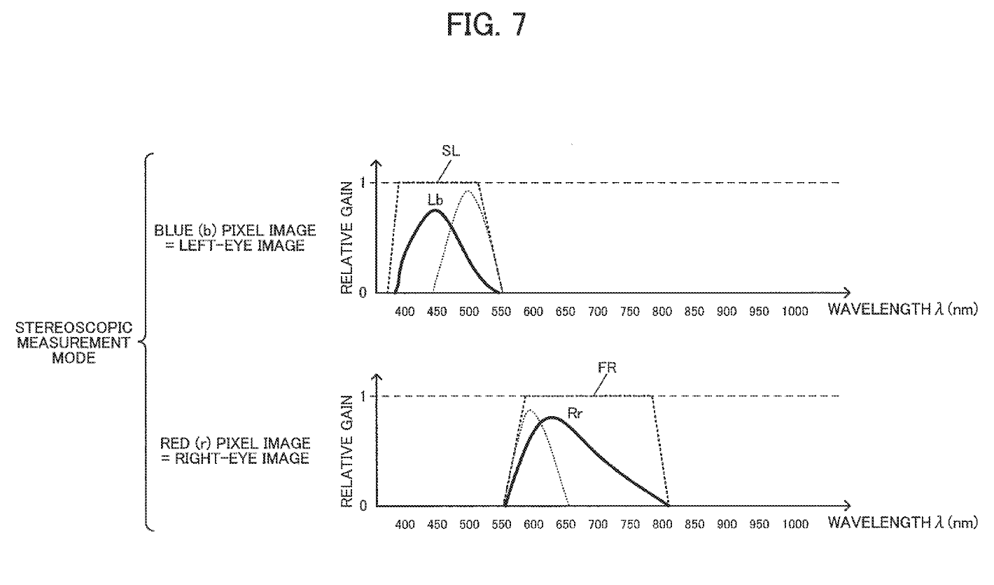

FIG. 7 illustrates spectral characteristics of the left-eye image, obtained with the image sensor 40 through the left-eye optical path, and the spectral characteristics of the right-eye image, obtained with the image sensor 40 through the right-eye optical path, during the stereoscopic measurement mode. In FIG. 7, the solid lines represent the spectral characteristics of the left-eye image and the right-eye image, the dotted lines represent the spectral characteristics SL of the left-eye optical path and the spectral characteristics FR of the right-eye optical path, and a small dotted line represents the spectral characteristics of a green image as a reference.

For example, the spectral image of the red color r obtained through a left-eye optical path L may be denoted with Lr, including the signs of the color and the path, for the sake of description. Similarly, other spectral images may be denoted with Lg, Lb, and Lir based on this rule. Generally, the image sensor 40 with RGB Bayer array has color pixels including: red pixels with a sensitive wavelength region (r+ir); green pixels with a sensitive wavelength region (g+ir); and blue pixels with a sensitive wavelength region (b+ir). Thus, in the observation mode, three types of color images Vr, Vg, and Vb represented by the following Formula (1) can be separately obtained. More specifically, Vr, Vg, and Vb respectively represent a red image, a green image, and a blue image (or their spectral characteristics) in the observation mode.

$$\begin{matrix}[ Formula 1 ] & \\ \begin{matrix}Vr = Lr + Lir \\ Vg = Lg + Lir \\ Vb = Lb + Lir\end{matrix} \} & ( 1 )\end{matrix}$$

In the stereoscopic measurement mode, two types of parallax images, obtained through the left-eye optical path and the right-eye optical path, are formed on the same image sensor 40 while being overlapped with each other, whereby a captured image involving image shift is acquired. The image shift corresponds to the amount of parallax with which depth information on the subject can be obtained, according to the principle of the stereoscopic measurement. To obtain the amount of parallax, the left-eye image and the right-eye image need to be separately obtained, and the phase difference needs to be detected by checking the correlation between the images (matching).

Thus, in the left-eye optical path in the measurement mode, the light with the spectral characteristics Lr, Lg, Lb, and Lir passing through the fixed mask 20 is filtered with the spectral characteristics SL of the left-eye optical path of the movable mask 30 so that light spectral characteristics Lb (including a part of the spectral characteristics Lg) is extracted. For example, as illustrated in FIG. 7, the spectral characteristics SL of the left-eye optical path of the movable mask 30 are set in such a manner that light with a wavelength not longer than 550 nm passes through and light with a wavelength not shorter than 550 nm is blocked.

Thus, in the right-eye optical the movable mask 30 is in the open state, and the light with the spectral characteristics Lr (partially including the spectral characteristics Lg) is extracted through filtering by the spectral characteristics FR of the light passing through the right-eye optical path of the fixed mask 20. For example, as illustrated in FIG. 7, the spectral characteristics FR of the right-eye optical path of the fixed mask 20 is set in such a manner that light with a wavelength not longer than 800 nm passes through and light with a wavelength not shorter than 550 nm is blocked.

Thus, in the stereoscopic measurement mode, the left-eye image from the left-eye optical path is obtained as an image with the spectral characteristics Lb due to the spectral characteristics of the blue pixels in the image sensor 40 (RGB Bayer array). Thus, the right-eye image from the right-eye optical path is obtained as an image with the spectral characteristics Lr due to the spectral characteristics of the red pixels in the image sensor 40 (RGB Bayer array). Thus, a left-eye image Mr and a right-eye image Mb, represented by Formula (2), can be separately obtained with color pixels different from each other. Specifically, Mr and Mb respectively represent a red image and a blue image (or their spectral characteristics) in the stereoscopic measurement mode. When the image sensor 40 supports complementary colors, complementary color information (cyan, magenta, yellow) may be converted so that the red image Mr and the blue image Mb are extracted.

$$\begin{matrix}[ Formula 2 ] & \\ \begin{matrix}Mr = Rr \\ Mb = Lb\end{matrix} \} & ( 2 )\end{matrix}$$

In the embodiment described above, an imaging device (endoscope apparatus) includes: the image sensor 40; the imaging optical system 10; the movable mask 30; and the second filter FR. The imaging optical system 10 (imaging optical device) forms an image of the subject 5 on the image sensor 40, by using the first optical path and the second optical path involving parallax relative to the first optical path. The movable mask 30 includes: the first filter SL transmitting light in a first wavelength band; and the light shielding section 34, and is movable relative to the imaging optical system 10. The second filter FR transmitting light in a second wavelength band different from the first wavelength band is provided in the second optical path.

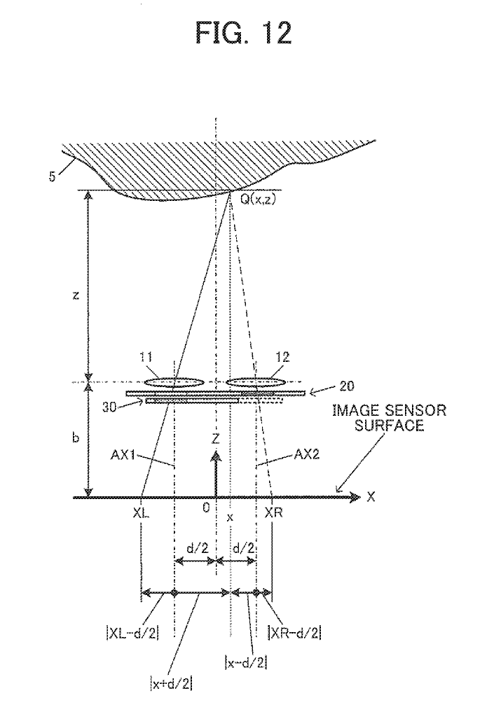

This configuration can achieve the switching between the observation mode and the stereoscopic measurement mode as described above with reference to FIG. 1 to FIG. 4. The parallax images in the color phase difference method can be simultaneously (non time-division manner) acquired, whereby stereoscopic measurement can be accurately performed. The movable mask 30 is provided as a single movable section. Thus, switching between the modes can be achieved at high speed, a simple driving mechanism, and with a lower risk of failure and error. The movable mask 30 can be achieved with a simple configuration obtained with a single filter SL provided to the light shielding section 34. Thus, a risk involved in the vibration due to the switching such as detaching of the filter can be reduced. The imaging system includes the first optical path and the second optical path clearly separated from each other, and thus can have a long baseline length (d in FIG. 12) for the stereoscopic measurement, whereby the distance measurement can be accurately performed.

For example, in the present embodiment, the first optical path corresponds to the left-eye optical path and the second optical path corresponds to the right-eye optical path. Alternatively, the first optical path may correspond to the right-eye optical path, and the second optical path may correspond to the left-eye optical path. The optical path is separated in the left and right direction for the sake of description, but the separation direction of the optical path is not limited to the left and right direction.

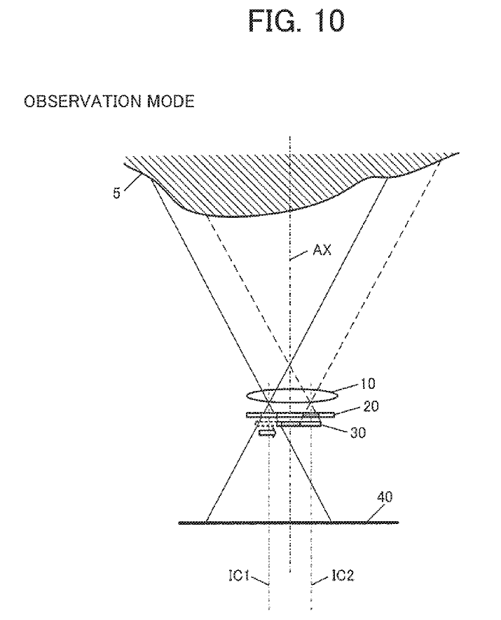

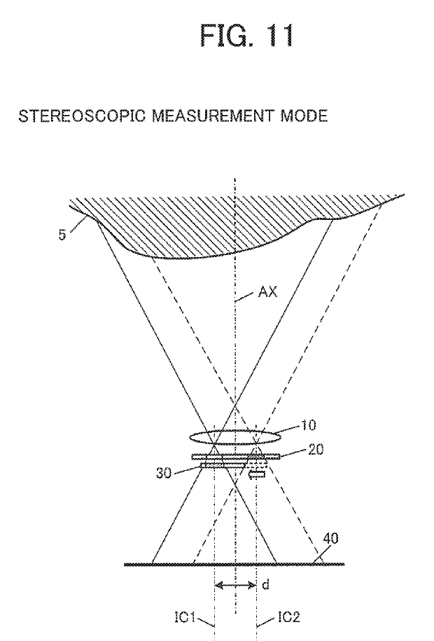

The optical path is a path through which light, corresponding to an image to be formed on the image sensor 40 and entered from an object side of the optical system, reaches the image sensor 40. In the configuration with the two optical systems described with reference to FIG. 1 to FIG. 4, the first optical path is an optical path that passes through the left-eye optical system 11 and the left-eye stop hole 21 of the fixed mask 20 (and also the stop hole 31 of the movable mask 30 in the stereoscopic measurement mode). The second optical path is an optical path that passes through the right-eye optical system 12 and the right-eye stop hole 22 of the fixed mask 20. Thus, optical paths are defined with the two optical systems and the two stop holes 21 and 22 of the fixed mask 20. In the configuration with a single optical system, described later with reference to FIG. 10 and FIG. 11, the first optical path is an optical path that passes through the imaging optical system 10 and the left-eye stop hole 21 of the fixed mask 20 (and also the stop hole 31 of the movable mask 30 in the stereoscopic measurement mode). The second optical path is an optical path that passes through the imaging optical system 10 and the right-eye stop hole 22 of the fixed mask 20. Thus, the optical paths are defined by dividing the pupil of the single optical system with the two stop holes 21 and 22 of the fixed mask 20.

The mask is a member or a component shielding light incident on the mask and also transmitting a part of the light. The fixed mask 20 and the movable mask 30 according to the present embodiment have the light shielding sections 24 and 34 that shield the light and the stop holes 21, 22, and 31 through which the light transmits (entire wavelength band, or a part of the entire wavelength band).

For example, in the present embodiment, the first wavelength band corresponds to the blue wavelength band (the wavelength band on the shorter wavelength side of the white light). The second wavelength band corresponds to the red wavelength band (the wavelength band on the longer wavelength side of the white light). Alternatively, the first wavelength band may correspond to the red wavelength band, and the second wavelength band may correspond to the blue wavelength band. The first wavelength band and the second wavelength band may be set in any way as long as the image obtained with the first optical path and the image obtained with the second optical path can be separated from each other based on the wavelength band. In the present embodiment, the blue image and the red image are separately obtained with the Bayer image sensor. However, this should not be construed in a limiting sense. The present invention may be applied to any method with which parallax images are separately obtained based on the wavelength band.

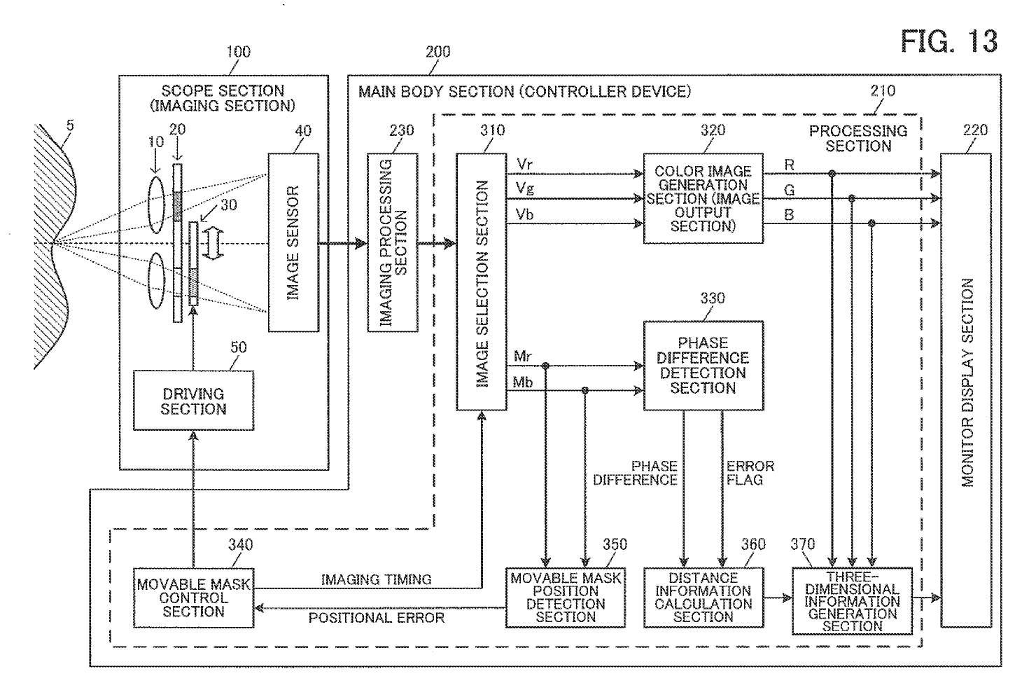

In the present embodiment, the imaging device includes a movable mask control section 340 that controls the movable mask 30 (FIG. 13). In the non-stereoscopic mode (observation mode), the movable mask control section 340 (processor) sets the movable mask 30 to be in the first state (at a first position) in which the first filter SL is not inserted in the first optical path and the light shielding section 34 is inserted in the second optical path. In a stereoscopic mode (stereoscopic measurement mode), the movable mask control section 340 sets the movable mask 30 to be in the second state (at the second position) in which the first filter SL is inserted in the first optical path and the light shielding section 34 is not inserted in the second optical path.

With such driving control for the movable mask 30, control for switching between the observation mode illustrated in FIG. 1 and FIG. 3 and the stereoscopic measurement mode illustrated in FIG. 2 and FIG. 4 can be achieved. Specifically, when the movable mask 30 is set to be in the first state, the second optical path is blocked by the light shielding section 34, and thus an image is captured only with the first optical path in which the first filter SL is not inserted. Thus, a normal observation image (white light image) can be captured. When the movable mask 30 is set to be in the second state, the first filter SL is inserted in the first optical path and the second filter FR is fixed to the second optical path. Thus, parallax images in the color phase difference method can be captured.

The imaging device according to the present embodiment includes the fixed mask 20. The fixed mask 20 includes the first stop hole 21 provided to the first optical path and the second stop hole 22 provided to the second optical path. The second filter FR is provided to the second stop hole 22. The movable mask 30 includes the third stop hole 31 provided to the light shielding section 34. The first filter SL is provided to the third stop hole 31.

With the fixed mask 20 thus including the first stop hole 21 and the second stop hole 22, the first optical path and the second optical path can be clearly separated from each other with the first stop hole 21 and the second stop hole 22. When the stop hole 22 as one of the holes is closed, an image can be captured with a single optical system in the observation mode. When both of the stop holes 21 and 22 are used, the stereoscopic imaging can be performed in the stereoscopic measurement mode. With the two optical paths clearly separated from each other with the fixed mask 20, the sufficient baseline length (d in FIG. 12) for the stereoscopic measurement can be ensured as described above.

In the present embodiment, the imaging optical system 10 includes: the first imaging optical system 11 that forms an image based on the light that has passed through the first stop hole 21; and the second imaging optical system 12 that forms an image based on the light that has passed through the second stop hole 22.

With the two optical systems in which the stop holes 21 and 22 can be provided on the optical axes, a high quality image (for example, an image with small aberrations) can be formed and obtained.

The imaging device according to the present embodiment may be configured as follows. Specifically, the imaging device according to the present embodiment includes: a memory that stores that stores information (for example, a program and various types of data); and a processor (processor including hardware) that operates based on the information stored in the memory. The processor performs a movable mask control process for controlling the movable mask 30. The movable mask control process includes setting, in the non-stereoscopic mode, the movable mask 30 to be in the first state, and setting, in the stereoscopic mode, the movable mask 30 to be in the second state.

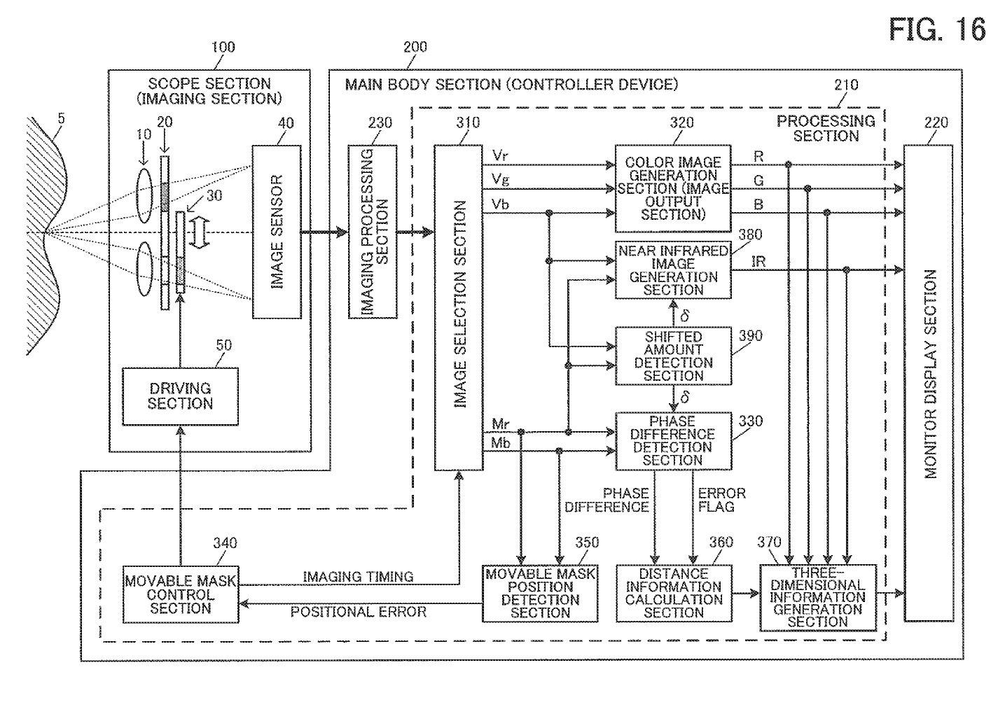

For example, the function of each section may be implemented by the processor or may be implemented by integrated hardware. For example, the processor may include hardware, and the hardware may include at least one of a circuit that processes a digital signal and a circuit that processes an analog signal. For example, the processor may include one or more circuit devices (e.g., IC), and one or more circuit elements (e.g., resistor or capacitor) that are mounted on a circuit board. The processor may be a central processing unit (CPU), for example. Note that the processor is not limited to a CPU. Various other processors such as a graphics processing unit (GPU) or a digital signal processor (DSP) may also be used. The processor may be a hardware circuit that includes an ASIC. The processor may include an amplifier circuit, a filter circuit, and the like that process an analog signal. The memory may be a semiconductor memory (e.g., SRAM or DRAM), or may be a register. The memory may be a magnetic storage device such as a hard disk drive (HDD), or may be an optical storage device such as an optical disc device. For example, the memory stores a computer-readable instruction, and the process (function) of each section of the imaging device is implemented by causing the processor to perform the instruction. As illustrated in FIG. 13 for example, sections of the imaging device include an imaging processing section 230, an image selection section 310, a color image generation section 320 (image output section), a phase difference detection section 330, a distance information calculation section 360, a movable mask position detection section 350, the movable mask control section 340, and a three-dimensional information generation section 370. As illustrated in FIG. 16, a near infrared image generation section 380 and a shifted amount detection section 390 are further provided in addition to the sections described above. The instruction may be an instruction set that is included in a program, or may be an instruction that instructs the hardware circuit included in the processor to operate.

For example, operations according to the present embodiment are implemented as follows. Specifically, the processor outputs a control signal, for setting the non-stereoscopic mode and setting the movable mask 30 to be in the first state, to a driving section 50. The driving section 50 sets the movable mask 30 to be in the first state. The processor also outputs a control signal, for setting the stereoscopic mode and setting the movable mask 30 to be in the second state, to a driving section 50. The driving section 50 sets the movable mask 30 to be in the second state.

The sections of the imaging device according to the present embodiment may be implemented as modules of a program operating on the processor. For example, the movable mask control section 340 is implemented as a movable mask control module that sets that movable mask 30 to be in the first state in the non-stereoscopic mode, and to be in the second state in the stereoscopic mode.

### 5. Modifications

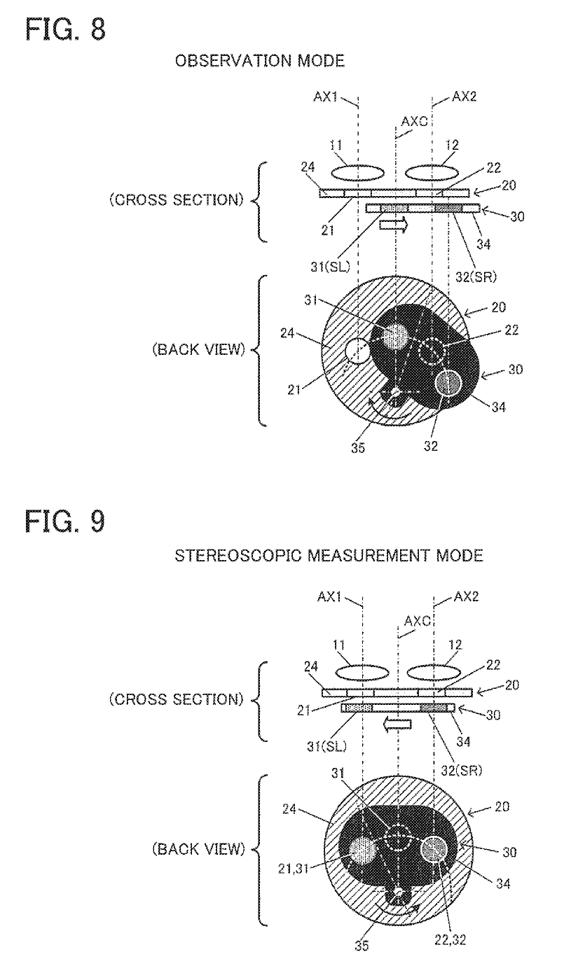

A first modification is described. The embodiment is described above with the movable mask 30 provided with a single stop hole 31 as an example. However, this should not be construed in a limiting sense. For example, as illustrated in FIG. 8 and FIG. 9, the movable mask 30 may be provided with two stop holes 31 and 32.

Specifically, the movable mask 30 includes: the light shielding section 34; and the left-eye stop hole 31 and the right-eye stop hole 32 provided to the light shielding section 34. The left-eye stop hole 31 is provided with a spectral filter having the spectral characteristics SL corresponding to the a short-wavelength. The right-eye stop hole 32 is provided with a spectral filter having the spectral characteristics SR corresponding to a long-wavelength. The spectral characteristics SR are the same as the spectral characteristics FR in FIG. 5.

The fixed mask 20 includes: the light shielding section 24; and the left-eye stop hole 21 and the right-eye stop hole 22 provided to the light shielding section 24. For example, the stop holes 21 and 22 are in the open state (through holes), and have the same spectral characteristics as the spectral characteristics FL in FIG. 5.

In the observation mode, the left-eye stop hole 21 of the fixed mask 20 is in the open state, and the right-eye stop hole 22 of the fixed mask 20 is shielded with the light shielding section 24 of the movable mask 30, whereby a white light image is captured with a single optical system. In the stereoscopic measurement mode, the left-eye stop hole 21 of the fixed mask 20 and the left-eye stop hole 31 of the movable mask 30 overlap, and the right-eye stop hole 22 of the fixed mask 20 and the right-eye stop hole 32 of the movable mask 30 overlap. Thus, parallax images (the red image and the blue image) in the color phase difference method are captured.

In this modification, an imaging device (endoscope apparatus) includes: the image sensor 40; the imaging optical system 10; and the movable mask 30. The imaging optical system 10 (imaging optical device) forms an image of the subject 5 on the image sensor 40, by using the first optical path and the second optical path involving parallax relative to the first optical path. The movable mask 30 includes: the first filter SL transmitting light in a first wavelength band (blue wavelength band); the second filter SR transmitting light in a second wavelength band (red wavelength band); and the light shielding section 34, and is movable relative to the imaging optical system 10.

In the present embodiment, the imaging device includes the movable mask control section 340 that controls the movable mask 30 (FIG. 13). In the non-stereoscopic mode (observation mode), the movable mask control section 340 sets the movable mask 30 to be in the first state in which the first filter SL is not inserted in the first optical path and the light shielding section 34 is inserted in the second optical path. In a stereoscopic mode (stereoscopic measurement mode), the movable mask control section 340 sets the movable mask 30 to be in the second state in which the first filter SL is inserted in the first optical path and the second filter SR is inserted in the second optical path.

This configuration can also achieve switching between the observation mode and the stereoscopic measurement mode, simultaneous acquisition of parallax images in the stereoscopic measurement mode, high speed mode switching, a simplified driving mechanism for the movable mask 30, and reduction of a risk of failure and error due to the mode switching, and can ensure the baseline length for the stereoscopic measurement.

Next, a second modification is described. Specifically, the embodiment is described above with the imaging optical system 10 including the two optical systems 11 and 12 as an example. However, this should not be construed in a limiting sense. For example, as illustrated in FIG. 10 and FIG. 11, the imaging optical system 10 may include a single optical system.

Specifically, this single imaging optical system 10 has the pupil divided with the stop holes 21 and 22 of the fixed mask 20, with a left pupil optical path serving as the left-eye optical path and a right pupil optical path serving as the right-eye optical path. For example, the stop holes 21 and 22 have centerlines (centerlines of the left pupil and the right pupil) IC1 and IC2 disposed at an equal distance from the optical axis AX of the single imaging optical system 10. The center lines IC1 and IC2 are preferably in the same plane but do not necessarily need to be in the same plane.

The imaging optical system 10 according to the present modification is a single imaging optical system that forms an image with light that has transmitted through the first stop hole 21 of the fixed mask 20 and light that has transmitted through the second stop hole 22 of the fixed mask 20.

With the imaging optical system 10 thus provided with the fixed mask 20, the stereographic imaging can be performed with the pupil divided. The fixed mask 20 can ensure the baseline length for the stereoscopic measurement. With a single optical system, a simpler configuration can be achieved.

### 6. Principle of Stereoscopic Three-Dimensional Measurement

The principle of the stereoscopic measurement in the stereoscopic measurement mode is described. As illustrated in FIG. 12, the optical paths for the left eye and the right eye are each independently formed. A reflected image from the subject 5 passes through these optical paths so that the subject image is formed on the image sensor surface (light receiving surface). A coordinate system X, Y, Z in the three-dimensional space is defined as follows. Specifically, an X axis and a Y axis orthogonal to the X axis are set along the image sensor surface. A Z axis, toward the subject, is set to be in a direction that is orthogonal to the image sensor surface, and parallel to the optical axes AX1 and AX2. The Z axis, the X axis, and the Y axis intersect at the zero point. The Y axis is omitted for the sake of illustration.

The left-eye imaging lens 11 and the right-eye imaging lens 12 are positioned at the same position on the Z axis. Here, the distance between the lenses 11, 12 and the image sensor surface is defined as b, and the distance between the lenses 11, 12 and a given point Q(x,z) of the subject 5 is defined as z. The optical axis centers AX1 and AX2 of the right-eye and the left-eye imaging lenses 11 and 12 are separated from the Z axis by the same distance d/2. Thus, the baseline length for the stereoscopic measurement is d. An X coordinate of a corresponding point, corresponding to the given point Q(x,y) of the subject 5, as a part of an image formed on the image sensor surface with the left-eye optical system 11 is XL. An X coordinate of the corresponding point, corresponding to the given point Q(x,y) of the subject 5, as a part of the image formed on the image sensor surface with the right-eye optical system 12 is XR. The following Formula (3) can be obtained based on a similarity relation among a plurality of partial right angle triangles formed within a triangle defined by the given point Q(x,z) and the coordinates XL and XR.

$$\begin{matrix}[ Formula 3 ] & \\ \frac{z}{b} = \frac{\vert x + d / 2 \vert}{\vert XL + d / 2 \vert} = \frac{\vert x - d / 2 \vert}{\vert XR - d / 2 \vert} & ( 3 )\end{matrix}$$

The following Formulae (4) and (5) hold true.

[Formula 4]

x+d/2>0 when XL+d/2<0

x+d/2<0 when XL+d/2>0 (4)

[Formula 5]

x−d/2>0 when XR−d/2<0

x−d/2<0 when XR−d/2>0 (5)

Thus, the absolute value in Formula (3) described above can be normal values as in the following Formula (6).

$$\begin{matrix}[ Formula 6 ] & \\ \frac{z}{b} = - \frac{x + d / 2}{XL + d / 2} = - \frac{x - d / 2}{XR - d / 2} & ( 6 )\end{matrix}$$

Formula (6) described above can be solved for x as in the Formula (7).

$$\begin{matrix}[ Formula 7 ] & \\ x = - \frac{d}{2} \cdot \frac{XR + XL}{XR - XL - d} & ( 7 )\end{matrix}$$

The following Formula (8) for obtaining z can be obtained by substituting x in Formula (7) described above into Formula (6) described above.

$$\begin{matrix}[ Formula 8 ] & \\ z = \frac{d}{( XR - XL - d )} \cdot b & ( 8 )\end{matrix}$$

Here, d and b are known setting values, and the unknown values XL and XR are obtained as follows. Specifically, the position XL on the image sensor surface is used as a reference (the pixel position in the left image is regarded as XL), and the position XR corresponding to the position XL is detected with matching processing (correlation calculation). The subject shape can be measured by calculating the distance z for each position XL. The distance z might be unobtainable in some cases due to matching failure. Such a distance z may be obtained by interpolation using the distances z obtained for the surrounding pixels or by other like method, for example.

### 7. Endoscope Apparatus

FIG. 13 illustrates an example of a configuration of an endoscope apparatus (an imaging device in a broad sense) according to the present embodiment. The scope section 100 (imaging section) includes a scope section 100 (imaging section) and a main body section 200 (controller device). The scope section 100 includes the imaging optical system 10, the fixed mask 20, the movable mask 30, the image sensor 40, and the driving section 50. The main body section 200 includes a processing section 210, a monitor display section 220, and an imaging processing section 230. The processing section 210 includes an image selection section 310 (image frame selection unit), a color image generation section 320 (image output section), a phase difference detection section 330, a movable mask control section 340 (movable mask driving controller section), a movable mask position detection section 350, a distance information calculation section 360, and the three-dimensional information generation section 370.

The main body section 200 may further include unillustrated elements such as an operation section for operating the main body section 200 and an interface section for connecting with external devices. The scope section 100 may further include unillustrated elements such as, for example, an operation section for operating the scope section 100, a treatment section, and an illumination section (a light source, a lens, and the like).

The endoscope apparatus may be what is known as a video scope (an endoscope apparatus incorporating an image sensor) for industrial and medical use. The present invention can be applied to a flexible endoscope with the scope section 100 that is flexible and to a rigid endoscope with the scope section 100 that is in a form of a stick. For example, a flexible endoscope for industrial use includes the main body section 200 and the imaging section 110 serving as a portable device that can be carried around. The flexible endoscope is used for inspection in manufacturing and maintenance processes for industrial products, in a maintenance process for buildings and pipes, and in other like situations.

The driving section 50 drives the movable mask 30 based on the control signal from the movable mask control section 340, to switch between the first state (observation mode) and the second state (stereoscopic measurement mode). For example, the driving section 50 includes an actuator including a piezoelectric element and a magnet mechanism.

The imaging processing section 230 executes an imaging process on a signal from the image sensor 40, and outputs a captured image (such as a Bayer image, for example). For example, a correlative double sampling process, a gain control process, an A/D conversion process, gamma correction, color correction, noise reduction, and the like are executed. For example, the imaging processing section 230 may include a discrete IC such as an ASIC, or may be incorporated in the image sensor 40 (sensor chip) and the processing section 210.

The monitor display section 220 displays an image captured by the scope section 100, three-dimensional shape information on the subject 5, or the like. For example, the monitor display section 220 includes a liquid crystal display, an Electro-Luminescence (EL) display, and the like.

An operation of the endoscope apparatus is described below. The movable mask control section 340 controls the driving section 50, and thus switches the position of the movable mask 30. When the movable mask control section 340 sets the movable mask 30 to be in the observation mode, an image of the subject 5 is formed on the image sensor 40 with reflected light from the subject 5 that has passed through the left-eye optical path. The imaging processing section 230 reads out pixel values of the image formed on the image sensor 40, performs the A/D conversion or the like, and outputs resultant image data to the image selection section 310.

The image selection section 310 detects that the movable mask 30 is in the state corresponding to the observation mode based on the control signal from the movable mask control section 340, and outputs {Vr, Vg, Vb}, selected from the captured image, to the color image generation section 320. The color image generation section 320 performs demosaicing process (process for generating an RGB image from a Bayer image) and various image processes, and outputs the resultant three-board RGB primary color image to the monitor display section 220. The monitor display section 220 displays this color image.

When the movable mask control section 340 sets the movable mask 30 to be in the stereoscopic measurement mode, images are simultaneously formed on the image sensor 40 based on the reflected light from the subject 5, through the left-eye optical path and the right-eye optical path. The imaging processing section 230 reads out pixel values of the image formed on the image sensor 40, performs the A/D conversion or the like, and outputs resultant image data to the image selection section 310.

The image selection section 310 detects that the movable mask 30 is in the state corresponding to the stereoscopic measurement mode based on the control signal from the movable mask control section 340, and outputs {Mr,Mb}, selected from the captured image, to the phase difference detection section 330. The phase difference detection section 330 executes a matching process on the two separate images Mr and Mb, to detect a phase difference (phase shift) for each pixel. The phase difference detection section 330 determines whether the detected phase difference is reliable, and outputs an error flag for each pixel determined to have an unreliable phase difference. Various matching evaluation methods for obtaining the amount of difference (phase difference) between two similar waveforms have conventionally been proposed, and thus can be used as appropriate. The proposed methods include normalized correlation calculation such as Zero-mean Normalized Cross-Correlation (ZNCC), and Sum of Absolute Difference (SAD) based on the sum of absolute differences between the waveforms.

Furthermore, parallax images Vr and Mr may be used for detecting phase shift (phase difference), but this method involves time division, which is negatively affected by movement of the subject and/or the imaging system. However, when the reflected light from the subject 5 includes a large amount of red components and a small amount of blue components, the measurement, which is difficult to perform with Mr and Mb, can be successfully performed with Vr and Mr both including the red components.

The phase difference detection section 330 outputs the phase difference information thus detected, and the error flag to the distance information calculation section 360. The distance information calculation section 360 calculates the distance information (for example, the distance z in FIG. 12) on the subject 5 for each pixel, and outputs the resultant distance information to the three-dimensional information generation section 370. For example, the pixel provided with the error flag may be regarded as a flat portion of the subject 5 (an area with a small amount of edge components), and interpolation may be performed for such pixel based on the distance information on surrounding pixels. The three-dimensional information generation section 370 generates three-dimensional information from the distance information (or from the distance information and the RGB image from the color image generation section 320). The three-dimensional information may be various types of information including a Z value map (distance map), polygon, and a pseudo-three-dimensional display image (with shape emphasized by shading or the like, for example). The three-dimensional information generation section 370 generates a three-dimensional image and three-dimensional data generated, or a display image obtained by superimposing the observation image on the image as appropriate, and outputs the resultant image and/or data to the monitor display section 220. The monitor display section 220 displays the three-dimensional information.

The movable mask position detection section 350 detects whether the movable mask 30 is at the position corresponding to the observation mode or at the position corresponding to the stereoscopic measurement mode by using the images {Mr,Mb } obtained in the stereoscopic measurement mode. When the movable mask 30 is in the state not matching the mode, a position error flag is output to the movable mask control section 340. Upon receiving the position error flag, the movable mask control section 340 corrects the movable mask 30 to be in the correct state (state corresponding to the image selection). For example, when the images {Mr,Mb } are determined to have no color shift even though the movable mask control section 340 is outputting the control signal for achieving the stereoscopic measurement mode, the movable mask 30 is actually at the position for the observation mode. In such a case, the correction is performed in such a manner that the position of the movable mask 30 matches the position indicated by the control signal. When the correction operation cannot achieve the correct state, some sort of failure is determined to have occurred, and thus the function of the entire system is stopped. For example, whether the movable mask 30 is at the position corresponding to the observation mode or is at the position corresponding to the stereoscopic measurement mode is detected or determined as follows.

Specifically, whether a position error has occurred is determined by matching the level (average level or the like) between determination areas in the image Mr and the image Mb, and then performing determination on a position error based on the sum of absolute difference between the image Mr and the image Mb (first method), determination based on correlation coefficients of the image Mr and the image Mb (second method), and the like.

In the first method, an absolute difference value between pixel values at each pixel is obtained, and the absolute values of all the pixels or a group of some of the pixels are integrated. When the resultant value exceeds a predetermined threshold, the image is determined to be that in the stereoscopic measurement mode. On the other hand, when the resultant value does not exceed the predetermined threshold, the image is determined to be that in the observation mode. In the stereoscopic measurement mode, basically, the image Mr and the image Mb have color misregistration, resulting in a predetermined difference value. Thus, the first method is performed based on this value.

In the second method, the correlation coefficient between the image Mr and the image Mb within a predetermined range is calculated. When the result of the calculation does not exceed a predetermined threshold, the image is determined as that in the stereoscopic measurement mode. When the result exceeds the predetermined threshold, the image is determined to be that in the observation mode. This method is based on the fact that the image obtained in the stereoscopic measurement mode has a small relative coefficient because the image Mr and the image Mb basically have color misregistration, and that the image Mr and the image Mb in the observation mode substantially match, and thus have a large relative coefficient.

### 8. Mode Switching Sequence

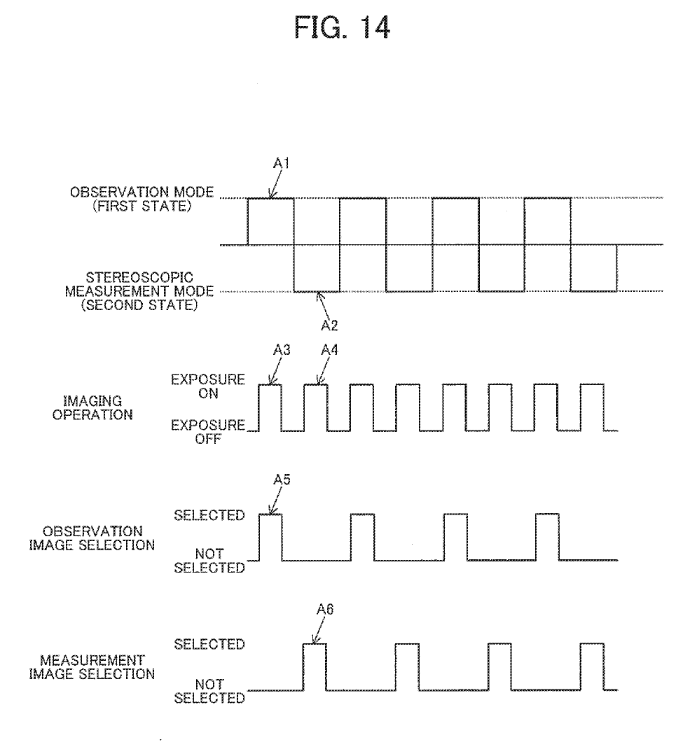

FIG. 14 illustrates a sequence for switching from the observation mode to the stereoscopic measurement mode in moving image capturing (operation timing chart).

With the stereoscopic measurement mode described above, accurate real time stereoscopic measurement can be performed even on a moving subject. However, the image obtained has color misregistration and thus cannot be used as a high level observation image This can be overcome by high speed switching between the observation mode and the stereoscopic measurement mode. Thus, the stereoscopic measurement can be executed while displaying the observation image in substantially real time.

As illustrated in FIG. 14, switching of the state of the movable mask 30, an image capturing timing, and selection of the captured image are interlocked. As indicated by A1 and A2, the mask state corresponding to the observation mode and the mask state corresponding to the stereoscopic measurement mode are alternately achieved. As indicated by A3 and A4, one image is captured in each mask state. As indicated by A5, the image captured with the image sensor 40 exposed in the mask state corresponding to the observation mode is selected as an observation image. As indicated by A6, an image captured with the image sensor 40 exposed in the stereoscopic measurement mode is selected as a measurement image.

With the observation mode and the stereoscopic measurement mode thus alternately repeated, the observation image and the measurement image can be contiguously obtained in substantially real time. Thus, the observing and the measurement can both be implemented even when the subject 5 is moving. When the image obtained in the observation mode is displayed with measurement information overlaid as appropriate, useful information can be provided so that the user can perform visual inspection and quantitative inspection at the same time.

In the present embodiment, in a first frame (A1 in FIG. 14), the movable mask control section 340 sets the non-stereoscopic mode (observation mode) and a first captured image (observation image) is obtained with the image sensor 40 (A3). Then, in a second frame (A2) subsequent to the first frame, the movable mask control section 340 sets the stereoscopic mode (stereoscopic measurement mode), and a second captured image (measurement image) is obtained with the image sensor 40 (A4).

Specifically, the imaging device (endoscope apparatus) alternately repeats the first frame (A1) and the second frame (A2) when the moving image is being captured. Thus, an operation that is the same as that in the first frame is performed in a third frame subsequent to the second frame.

More specifically, the imaging device includes: an image output section (the color image generation section 320, which is a processor) that outputs a moving image for observation; and a phase difference detection section 330 (processor) that detects a phase difference between the image (blue image Mb) corresponding to the first wavelength band and the image (red image Mr) corresponding to the second wavelength band based on the second captured image in the moving image.

With the moving image captured by alternately repeating the image capturing in the observation mode and the image capturing in the stereoscopic measurement mode, the real time stereoscopic measurement can be performed for the subject 5 while performing the observation with a normal image obtained with a single optical system. The configuration according to the present embodiment features the movable mask 30 and the fixed mask 20 suitable for high speed switching, and thus is suitable for the real time measurement.

In the present embodiment, the imaging device includes the movable mask position detection section 350. The movable mask position detection section 350 (processor) detects whether the movable mask 30 is set to be in the second state in the stereoscopic mode, based on a similarity (based on the sum of absolute differences, the correlation coefficient, or the like described above with reference to FIG. 13) between the image (blue image Mb) corresponding to the first wavelength band and the image (red image Mr) corresponding to the second wavelength band in the image captured in the stereoscopic mode.

When it is determined that the movable mask 30 is set in the first state in the stereoscopic mode, the movable mask control section 340 (processor) corrects the movable mask 30 so that the state and the mode match.

The mechanical movable member such as the movable mask 30 might not actually operate as indicated by the control due to factors such as operation failure, for example. When such an error occurs, an image with color misregistration might be displayed as the observation image, or appropriate stereoscopic measurement might not be achievable. In view of this, in the present embodiment, whether the mask state matches the mode can be determined based on the similarity between the parallax images. Thus, the mask state can be corrected to match the mode based on the result of the determination. In the observation mode, a red image and a blue image are captured with a single optical system, and thus involve no phase difference and have high similarity. Thus, the movable mask 30 can be determined to be erroneously at the position corresponding to the observation mode, when a red image and a blue image having high similarity are obtained in the stereoscopic measurement mode.

### 9. Near Infrared Image Extraction Process

As described above, in the present embodiment, the observation image can be captured and the stereoscopic measurement can be performed. Furthermore, a near infrared image can be obtained by using captured images obtained in the modes. A method for acquiring the near infrared image is described below.

Specifically, the near infrared image can be extracted with the following Formula (9) based on Formulae (1) and (2) described above. The image Vb=Lb+Lir in the observation mode and the image Mb=Lb in the stereoscopic measurement mode are not simultaneously obtained, and thus the definition Mb′=Lb′ can be used for distinction. The images are both obtained from light through the left-eye optical path, and thus are expected to have no phase difference. Furthermore, the blue images Lb and Lb′ with the same spectral characteristics are expected to have high similarity. Thus, an image including a near infrared component Lir can be generated with the blue component Lb eliminated from the image Vb=Lb+Lir based on the definition image Mb′=Lb′, as in the following Formula (9).

[Formula 9]

Vb−Mb′=Lb+Lir−Lb′=Lir (9)

### 10. Countermeasure Against Movement of Subject or Imaging System

When the subject or the imaging system moves while a near infrared image is being extracted, the similarity between the blue image Lb and the blue image Lb′ becomes low. Countermeasure against this is described below.

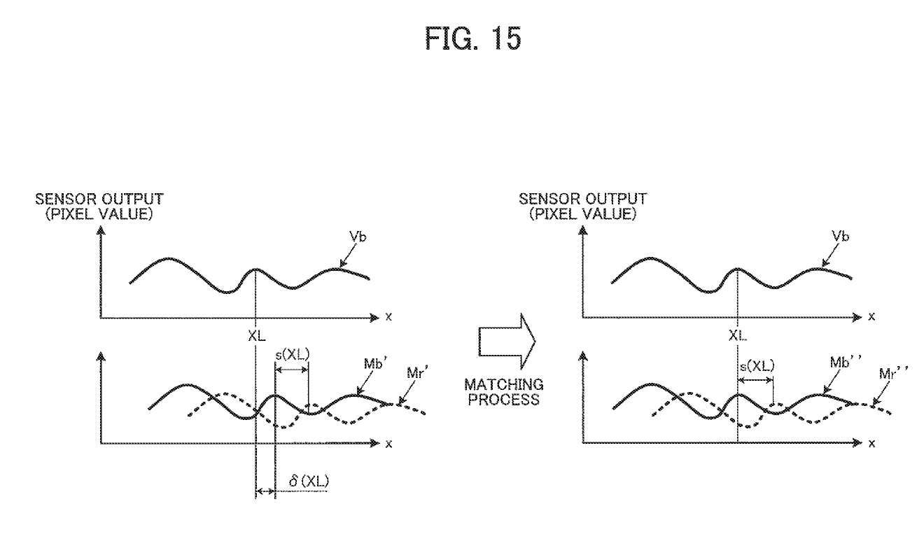

The image Vb, obtained in the observation mode, and the image Mb′, obtained in the measurement mode, are captured at different timings. Thus, when the subject or the imaging system moves during the interval between the timings, positional shift occurs between the images. FIG. 15 illustrates a case where a pixel corresponding to the coordinate position XL in the image Vb is shifted by a shifted amount δ(XL) on the image Mb′. The image Vb and the image Mb′ are obtained with slightly different viewpoints. Thus, the shifted amount δ(XL) not only includes a shifted amount due to the movement but also actually includes the amount of parallax (shifting between images due to different view directions), which depends on a distance to the subject reflection surface. Thus, the shifted amount needs to be obtained for each coordinate position XL in the image Vb, and is denoted with δ(XL).

The shifted amount δ(XL) can be obtained by a matching search process around the coordinate position XL in the image Vb and the image Mb′. The matching points might not necessarily be obtained at all the coordinate positions due to factors such as image quality deterioration and the like. Thus, only reliable δ(XL) is deemed as usable. A coordinate position where no δ(XL) is obtained is set as an immeasurable point. The distance z to the subject reflection surface is not calculated for the immeasurable point, in the phase difference detection in a later stage. The immeasurable point may be treated as appropriate. For example, a user may be notified of information indicating the immeasurable point, or the immeasurable point may be interpolated based on surrounding measurement values.

The image Mb′ and the image Mr′ are shifted relative to each other by the shifted amount δ(XL), based on the usable δ(XL). Thus, a positional displacement is corrected. Images obtained by correcting the positional shift are denoted with Mb″ and Mr″. Then, a phase difference amount s(XL) for the measurement is obtained at the coordinate position XL through the matching search process between the image Mb″ and the image Mr″. When the phase difference amount s(XL) is obtained, the distance z to the subject reflection surface corresponding to the coordinate position XL can be obtained through a method described with reference to FIG. 12.

### 11. Second Configuration Example of Endoscope Apparatus

FIG. 16 illustrates a configuration example of an endoscope apparatus additionally provided with a function of capturing a near infrared image. The endoscope apparatus includes the scope section 100 and the main body section 200. The scope section 100 includes the imaging optical system 10, the fixed mask 20, the movable mask 30, the image sensor 40, and the driving section 50. The main body section 200 includes a processing section 210, a monitor display section 220, and an imaging processing section 230. The processing section 210 includes the image selection section 310, the color image generation section 320, the phase difference detection section 330, the movable mask control section 340, the movable mask position detection section 350, the distance information calculation section 360, the three-dimensional information generation section 370, a near infrared image generation section 380, and a shifted amount detection section 390. The components that are the same as those already described above are denoted with the same reference signs, and the description thereof is omitted as appropriate.

The image selection section 310 outputs the image Vb and the image Mb (Mb′ in FIG. 15) to be input to the near infrared image generation section 380. The near infrared image generation section 380 calculates the image Vb and the image Mb with Formula (9) described above, and obtains an image Lir including the near infrared component from the left-eye optical system. The near infrared image generation section 380 performs image processes on the image Lir thus obtained, and outputs the resultant near infrared image IR. The near infrared image IR is input to the monitor display section 220 and thus can be displayed.

The color image generation section 320 performs image processes on the observation image {Vr, Vg, Vb} to output a color image {R, G, B} that can be displayed. The color image {R, G, B} is input to the monitor display section 220, so that the colored observation image can be displayed.

The color image {R,G,B} and the near infrared image IR are both obtained with the left-eye optical path, and thus involve no image shifting and positional displacement. Thus, the images can be easily integrated to be displayed by being overlapped with each other, and the near infrared image can be easily colored by using color information on the color image.

The three-dimensional information and the near infrared image IR may be associated with each other by the three-dimensional information generation section 370. Thus, a three-dimensional near infrared image can be obtained with the distance information associated with each pixel of the near infrared image IR. Thus, an observation color image as a visible image, a near infrared observation image, and the distance information can be substantially simultaneously obtained, whereby information can be presented in various modes.

In the embodiment described above, the imaging device (endoscope apparatus) includes the near infrared image generation section 380. The near infrared image generation section 380 (processor) generates the near infrared image Lir (or IR) based on the first captured image {Vr, Vg, Vb} captured in the non-stereoscopic mode (observation mode) and the second captured image {Mr, Mb} captured in the stereoscopic mode (stereoscopic measurement mode).

Specifically, the captured image obtained with the image sensor 40 includes images of the red color r, the green color g, and the blue color b. The first wavelength band SL corresponds to one of the red color r and the blue color b. The second wavelength band FR corresponds to the other one of the red color r and the blue color b. The near infrared image generation section 380 generates the near infrared image Lir (or IR) based on a difference between the red image Vr of the first captured image and the red image Mr of the second captured image or a difference between the blue image Vb of the first captured image and the blue image Mb of the second captured image.

For example, in the present embodiment, the first wavelength band SL is a blue wavelength band (the band SL corresponding to the characteristics Lb in FIG. 5). The second wavelength band FR is the red wavelength band (the band FR corresponding to the characteristics Rr in FIG. 5). However, this should not be construed in a limiting sense, and the opposite combination between the bands and the colors may be employed For example, in the present embodiment, the near infrared image Lir is obtained based on the difference between the blue images Vb and Mb (Formula (9) described above). However, this should not be construed in a limiting sense, and the near infrared image Lir may be obtained based on the difference between the red images Vr and Mr.

The image senor 40 generally has a sensitivity covering the near infrared band, and thus the white light image captured in the observation mode includes a near infrared image. However, in the stereoscopic measurement mode involving the red and the blue spectral filters, the near infrared band is almost entirely filtered out, and thus an image including substantially no near infrared image is obtained. Thus, the near infrared image can be extracted based on the difference between the images. With the near infrared image thus acquired, an object that cannot be observed (difficult to observe) with visible light might be observable.

The embodiments and the modifications thereof according to the present invention are described. However, the present invention is not limited the embodiments and the modifications only, and thus can be implemented with the elements modified without departing from the gist of the invention. The plurality of elements disclosed in the embodiments and the modifications may be combined as appropriate to implement the invention in various ways. For example, some of all the elements described in the embodiments and the modifications may be deleted. Furthermore, elements in different embodiments and modifications may be combined as appropriate. Thus, various modification and application can be made without departing from the gist of the present invention. The terms (observation mode, stereoscopic measurement mode, left-eye optical path, right-eye optical path, and the like) that is at least once written as the other term with a broader concept or a narrower concept (non-stereoscopic mode, stereoscopic mode, first optical path, second optical path, and the like) can be replaced with the other terms in any portion of the specification or figures.

What is claimed is:

1. An imaging device comprising: an image sensor;
an imaging optical device forming an image of a subject on the image sensor by using a first optical path and a second optical path involving parallax with respect to the first optical path;
a movable mask including a first filter transmitting light in a first wavelength band and a light shielding section, the movable mask being movable relative to the imaging optical device; and
a second filter transmitting light in a second wavelength band different from the first wavelength band, the second filter being provided to the second optical path.

2. The imaging device as defined in claim 1,
further comprising a processor comprising hardware, the processor being configured to implement:
a movable mask control process that controls the movable mask,
the processor implementing the movable mask control process including:
setting, in a non-stereoscopic mode, the movable mask to be in a first state in which the first filter is not inserted in the first optical path and the light shielding section is inserted in the second optical path, and
setting, in a stereoscopic mode, the movable mask to be in a second state in which the first filter is inserted in the first optical path and the light shielding section is not inserted in the second optical path.

3. The imaging device as defined in claim 2,
the processor implementing the movable mask control process including setting the non-stereoscopic mode, the image sensor capturing a first captured image, in a first frame, and
the processing implementing the movable mask control process including setting the stereoscopic mode, the image sensor capturing a second captured image, in a second frame subsequent to the first frame.

4. The imaging device as defined in claim 3,
the first frame and the second frame being alternately repeated when a moving image is captured.

5. The imaging device as defined in claim 4,
the processor being configured to implement:
an image output process that outputs an observation moving image based on the first captured image in the moving image; and
a phase difference detection process that detects a phase difference between an image corresponding to the first wavelength band and an image corresponding to the second wavelength band, based on the second captured image in the moving image

6. The imaging device as defined in claim 2,
the processor being configured to implement:
a movable mask detection process that detects whether the movable mask is set to be in the second state in the stereoscopic mode, based on a similarity between an image corresponding to the first wavelength band and an image corresponding to the second wavelength band that are in an image captured in the stereoscopic mode.

7. The imaging device as defined in claim 6,
the processor being configured to implement:
the movable mask control process including
performing correction, when the movable mask is detected to be in the first state in the stereoscopic mode, in such a manner that correct relationship between a state of the movable mask and the mode match.

8. The imaging device as defined in claim 2,
the processor being configured to implement:
a near infrared image generation process that generates a near infrared image based on the first captured image captured in the non-stereoscopic mode and the second captured image captured in the stereoscopic mode.

9. The imaging device as defined in claim 8,
the captured image obtained by the image sensor including images of a red color, a green color, and a blue color,
the first wavelength band corresponding to one of the red color and the blue color,
the second wavelength band corresponding to another one of the red color and the blue color,
the processor being configured to implement:
the near infrared image generation process including
generating the near infrared image based on a difference between the image of the red color in the first captured image and the image of the red color in the second captured image or based on a difference between the image of the blue color in the first captured image and the image of the blue color in the second captured image.

10. The imaging device as defined in claim 1,
a fixed mask including a first stop hole provided to the first optical path and a second stop hole provided to the second optical path,
the second filter being provided to the second stop hole,
the movable mask including a third stop hole being provided to the light shielding section,
the first filter being provided to the third stop hole.

11. The imaging device as defined in claim 10,
the imaging optical device including:
a first imaging optical device forming an image from light that has passed through the first stop hole; and a second imaging optical device forming an image from light that has passed through the second stop hole.

12. The imaging device as defined in claim 10,
the imaging optical device including:
the imaging optical device being a single-lens imaging optical device forming an image from light that has passed through the first stop hole and light that has passed through the second stop hole.

13. The imaging device as defined in claim 1,
further comprising a processor comprising hardware, the processor being configured to implement:
a phase difference detection process that detects a phase difference between an image corresponding to the first wavelength band and an image corresponding to the second wavelength band, based on an image captured under a condition that the movable mask is set to be in a state in which the first filter is inserted in the first optical path and the light shielding section is not inserted in the second optical path.

14. The imaging device as defined in claim 1,
the captured image obtained by the image sensor including images of a red color, a green color, and a blue color,
the first wavelength band corresponding to one of the red color and the blue color,
the second wavelength band being a wavelength band corresponding to another one of the red color and the blue color.

15. An imaging device comprising: an image sensor;
an imaging optical device forming an image of a subject on the image sensor by using a first optical path and a second optical path involving parallax with respect to the first optical path;
a movable mask including a first filter transmitting light in a first wavelength band, a second filter transmitting light in a second wavelength band, and a light shielding section, the movable mask being movable relative to the imaging optical device.

16. The imaging device as defined in claim 15,
further comprising a processor comprising hardware, the processor being configured to implement:
a movable mask control process that controls the movable mask,
the processor implementing the movable mask control process including:
setting, in a non-stereoscopic mode, the movable mask to be in a first state in which the first filter is not inserted in the first optical path and the light shielding section is inserted in the second optical path, and
setting, in a stereoscopic mode, the movable mask to be in a second state in which the first filter is inserted in the first optical path and the second filter is inserted in the second optical path.

17. An endoscope apparatus comprising the imaging device as defined in claim 1.

18. An endoscope apparatus comprising the imaging device as defined in claim 15.

19. An imaging method comprising: setting, in a non-stereoscopic mode, a movable mask including a first filter transmitting light in a first wavelength band and a light shielding section in a first state, in such a manner that the first filter is not inserted in a first optical path of an imaging optical device and the light shielding section is inserted in a second optical path of the imaging optical device, the second optical path involving parallax with respect to the first optical path;
and setting, in a stereoscopic mode, the movable mask to be in a second state in such a manner that the first filter is inserted in the first optical path and the light shielding section is not inserted in the second optical path.
